(19) United States
(12) Patent Application Publication
(10) Pub. No.: US 2020/0199580 A1
(43) Pub. Date: Jun. 25, 2020

(54) METHOD FOR HIGH-THROUGHPUT DETECTION OF DIFFERENTIAL EXPRESSION OF PLANT CIRCRNA ALLELIC LOCI

(71) Applicant: Beijing Forestry University, Beijing (CN)

(72) Inventors: Deqiang ZHANG, Beijing (CN); Yuepeng SONG, Beijing (CN); Anran XUAN, Beijing (CN); Chenhao BU, Beijing (CN)

(73) Assignee: Beijing Forestry University, Beijing (CN)

(21) Appl. No.: 16/585,766

(22) Filed: Sep. 27, 2019

(30) Foreign Application Priority Data
Dec. 24, 2018 (CN) 201811582470.8

(51) Int. Cl. C12N 15/10; G16B 20/20; G16B 30/10

(52) U.S. Cl. CPC C12N 15/1072; G16B 30/10; G16B 20/20

(57) ABSTRACT

The invention relates to the technical field of gene expression detection, and provides methods for high-throughput detection of differential expression of plant circRNA allelic loci. The method comprises the following steps: 1) extracting total RNA of a plant sample, and constructing a strand-specific library; 2) performing paired-end sequencing on the strand-specific library with Illumina HiSeq; 3) screening circRNAs data from raw sequencing data; 4) extracting reverse splicing reads at a circRNAs looping position to form the circRNAs data; 5) performing single nucleotide variation detection on the reverse splicing reads; and 6) collecting statistics about the number of reads of different genotypes, which are aligned to SNP loci, in the reverse splicing reads, to align the proportion of the number of reads of different genotypes to the proportion of expression quantities of different genotypes. The methods can accurately detect differential expression of circRNA allelic loci with high throughput.

### CROSS-REFERENCE TO RELATED APPLICATIONS AND CLAIM TO PRIORITY

This application claims priority to Chinese application number 201811582470.8, filed Dec. 24, 2018 with a title of METHOD FOR HIGH-THROUGHPUT DETECTION OF DIFFERENTIAL EXPRESSION OF PLANT CIRCRNA ALLELIC LOCI, which is incorporated herein by reference in its entirety.

### FIELD OF THE INVENTION

The present invention relates to the technical field of gene expression detection, and in particular, to methods for high-throughput detection of differential expression of plant circRNA allelic loci.

### BACKGROUND OF THE INVENTION

An allele (also called allelomorph) generally refers to a pair of genes that regulate phenotypic traits at the same position on homologous chromosomes

Allelic Expression Imbalance (AEI) is in the same cell, and each gene generally has two copies. The cis-acting or trans-acting shifts the expression ratio of the two copies of the gene by 1:1. The phenomenon of AEI is ubiquitous. In addition to the absolute imbalance expression of genetic imprinted gene, a considerable number of genes have AEI in different individuals and in the same individual at different developmental phase and tissues. It is also associated with polymorphic loci in specific regions of the genome.

At present, the commonly used AEI detection mainly focuses on the genes coding for proteins. However, there is no high-throughput accurate analysis method for the allelic expression of circRNA widely existed in transcriptome data.

### SUMMARY OF THE INVENTION

In view of the above, an objective of the present invention is to provide a method for high-throughput detection of differential expression of plant circRNA allelic loci.

To achieve the above purpose, the present invention provides the following technical solution.

A method for high-throughput detection of differential expression of plant circRNA allelic loci, which includes the following steps:

1) extracting total RNA of a plant sample, and constructing a strand-specific library with the total RNA;

2) performing paired-end sequencing on the strand-specific library in step 1) with Illumina HiSeq to obtain raw sequencing data;

3) screening circRNAs data from the raw sequencing data obtained in step 2);

4) extracting reverse splicing reads at a circRNAs looping position from the circRNAs data obtained in step 3);

5) performing single nucleotide variation detection on the reverse splicing reads to obtain SNP loci in the reverse splicing reads; and

6) collecting statistics about the number of reads of different genotypes, which are aligned to the SNP loci in step 5, in the reverse splicing reads in step 4, to align the proportion of the number of reads of different genotypes to the proportion of expression quantities of different genotypes.

Preferably, the screening circRNAs data in step 3) includes the following steps: 3.1) performing transcript splicing on the raw sequencing data according to a reference genome;

3.2) extracting 18-22 nt (nucleotides) from both ends of each read in the raw sequencing data that is not aligned to the reads on the reference genome, and forming a pair of anchor sequences, the anchor sequence including a 5′-terminal sequence and a 3′-terminal sequence;

3.3) re-aligning the anchor sequence to a reference genome, where the 5′-terminal sequence of the anchor sequence is aligned to the 3′-terminal of the reference sequence, and the 3′-terminal sequence of the anchor sequence is aligned to the upstream of a matching locus of the 5′-terminal sequence of the anchor sequence in the reference sequence, and a splicing locus GT-AG exists between the matching locus of the 5′-terminal sequence of the anchor sequence and a matching locus of the 3′-terminal sequence of the anchor sequence in the reference sequence, and this splicing locus read is used as circRNA data.

Preferably, the screening circRNAs data is implemented by means of the find_circ software and the CIRIexplorer software.

Preferably, the circRNAs are screened by using the find_circ software and the CIRIexplorer software, respectively, to obtain candidate data of circRNAs screened by the find_circ software and candidate data of circRNAs screened by the CIRIexplorer software, and an intersection of the candidate data of circRNAs screened by the find_circ software and the candidate data of circRNAs screened by the CIRIexplorer software is used as the circRNAs data.

Preferably, the extracting reverse splicing reads at a circRNAs looping position in the circRNAs obtained in step 4) is implemented using a samtools view—R instruction in the find_circ software.

Preferably, the single nucleotide variation detection in step 5) is performed using a SNP calling in the GATK software.

Preferably, after extracting total RNA of a plant sample and before constructing a strand-specific library in step 1), the method further includes a step of removing rRNA and a step of linear RNA digestion that are performed sequentially.

Preferably, a reaction system of the linear RNA digestion is 50 μL, including the following components: 5 μg of RNA, 5 μL of 10× Reaction Buffer, 20 U of RNase R, and the balance of RNase-Free water.

Preferably, the temperature for the linear RNA digestion is 36-38° C., and the time for the linear RNA digestion is 1-2 h.

Preferably, the plant is a forest tree.

The advantageous effects of the present invention: the method of the present invention can perform high-throughput and accurate analysis of differential expression of allelic loci for circRNAs widely existing in transcriptome data and provide a new research strategy for systematic analysis of the transcriptome data.

### BRIEF DESCRIPTION OF THE DRAWINGS

FIG. 1 is a flowchart showing the differential expression analysis of plant circRNA allelic loci.

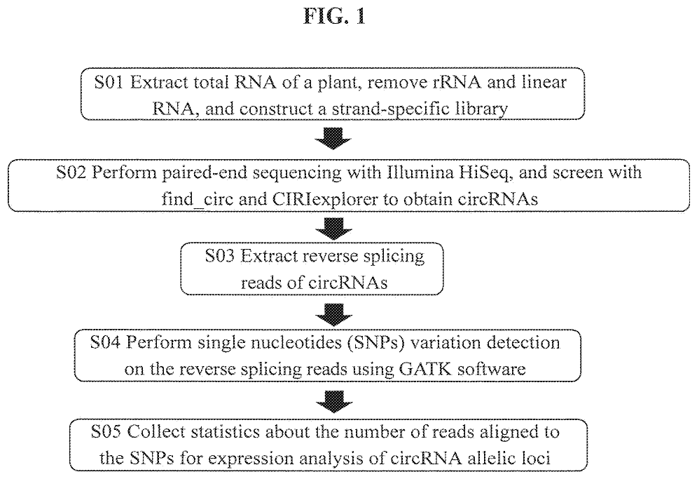

### DETAILED DESCRIPTION OF THE PREFERRED EMBODIMENTS

The present invention provides a method for high-throughput detection of differential expression of plant circRNA allelic loci, including the following steps:

1) total RNA of a plant sample is extracted, and a strand-specific library is constructed with the total RNA;

2) paired-end sequencing is performed on the strand-specific library in step 1) with Illumina HiSeq to obtain raw sequencing data;

3) circRNAs data is screened from the raw sequencing data obtained in step 2);

4) reverse splicing reads at a circRNAs looping position are extracted from the circRNAs data obtained in step 3);

5) single nucleotide variation detection is performed on the reverse splicing reads to obtain SNP loci in the reverse splicing reads; and

6) the number of reads of different genotypes, which are aligned to the SNP loci in step 5, in the reverse splicing reads in step 4 are counted to align the proportion of the number of reads of different genotypes to the proportion of expression quantities of different genotypes.

The present invention extracts total RNA of the plant sample and constructs a strand-specific library with the total RNA. The present invention has no special requirement for the types of plant samples, and conventional plants may be used, preferably forest trees. In the specific implementation process of the present invention, Populus tomentosa is selected. The plant sample in the present invention is preferably a leaf tissue. The present invention has no particular limitation on the method for extracting total RNA of the plant sample, and the conventional total RNA extraction method in the art may be used. In the specific implementation process of the present invention, the total RNA is extracted by using an RNA extraction kit (e.g., MagJ ET Plant RNA Purification Kit, No. K2772).

After extracting the total RNA and before constructing the strand-specific library, the present invention preferably further includes a step of removing rRNA and a step of linear RNA digestion that are performed sequentially. The step of removing rRNA is preferably performed by using Ribo-Zero™ rRNA Removal Kits (Plant) (No. MRZPL116). In the present invention, the method for removing rRNA preferably includes: mixing 30-50 μl of total RNA with 50-70 μl of magnetic beads, vortexing for 8-12 s, standing at room temperature for 4-6 min, incubating at 49-51° C. for 4-6 min, placing on a magnetic frame until a supernatant is clarified, and collecting the supernatant; and more preferably, mixing 40 μl of total RNA with 60 μl of magnetic beads, vortexing for 10 s, standing at room temperature for 5 min, incubating at 50° C. for 5 min, placing on a magnetic frame until a supernatant is clarified for 2 min, and collecting the supernatant.

The present invention obtains a Poly (A)-RNA sample, i.e., a linear RNA, after the step of removing the rRNA, preferably digests the obtained linear RNA. The digestion is preferably performed by using RNase Rd. A reaction system of the linear RNA digestion is preferably 50 μL, including the following components: 5 μg of RNA, 5 μL of 10× Reaction Buffer, 20 U of RNase R, and the balance of RNase-Free water. The temperature for the linear RNA digestion is preferably 36-38° C., and more preferably 37° C. The time for the linear RNA digestion is preferably 1-2 h, and more preferably 1.5 h. In the present invention, after the digestion, a strand-specific library is constructed using the digested RNA. In the present invention, a SMART Kit (SMART cDNA Library Construction Kit, NO. 634901) is preferably used to construct the strand-specific library.

After obtaining the strand-specific library, the present invention performs paired-end sequencing on the strand-specific library with Illumina HiSeq to obtain raw sequencing data. The read length of the sequencing described in the present invention is preferably 150 nt. The amount of data for sequencing is preferably greater than 12 G. The sequencing in the present invention may be entrusted to Novogene Company.

After obtaining the raw sequencing data, the present invention screens circRNAs data from the obtained raw sequencing data. In the particular implementation process of the present invention, a linker and the redundant sequence in the raw sequencing data are first removed. In the present invention, the screening circRNAs data includes the following steps:

3.1) transcript splicing is performed on the raw sequencing data;

3.2) 18-22 nt are extracted from both ends of each read in the raw sequencing data that is not aligned to the reads on the reference genome, and a pair of anchor sequences is formed, the anchor sequence including a 5′-terminal sequence and a 3′-terminal sequence; and

3.3) the anchor sequence is re-aligned to a reference genome, where the 5′-terminal sequence of the anchor sequence is aligned to the 3′-terminal of the reference sequence, and the 3′-terminal sequence of the anchor sequence is aligned to the upstream of a matching locus of the 5′-terminal sequence of the anchor sequence in the reference sequence, and a splicing locus GT-AG exists between the matching locus of the 5′-terminal sequence of the anchor sequence and a matching locus of the 3′-terminal sequence of the anchor sequence in the reference sequence, and this splicing locus read is used as circRNA data.

In the present invention, the transcript splicing is performed preferably using default parameters of the Cufflinks software. Steps 3.2) and 3.3) are preferably implemented by the find_circ software and the CIRIexplorer software. More preferably, the circRNAs are screened by using the find_circ software and the CIRIexplorer software, respectively, to obtain candidate data of circRNAs screened by the find_circ software and candidate data of circRNAs screened by the CIRIexplorer software, and an intersection of the candidate data of circRNAs screened by the find_circ software and the candidate data of circRNAs screened by the CIRIexplorer software is used as the circRNAs data.

In the specific implementation process of the present invention, the screening parameters of the find_circ software and the CIRIexplorer software for screening circRNAs include -q 5, -a 20, -m 2, -d 2, and --noncanonical. The screening criteria of the foregoing parameters are preferably: (1) -q 5: the minimum support number of anchor sequence alignment is 5; (2) -a 20: the anchor sequence is 20 bp; (3) -m 2: a branch point cannot occur elsewhere in the range of two nucleic acids in the anchor sequence; (4) -d 2: sequence alignment only supports two mismatches; and (5) --noncanonical: GU/AG appears on both sides of a splicing locus and a clear breakpoint can be detected.

Since the find_circ software and the CIRIexplorer software generate false positive data in the process of screening circRNAs, the intersection of the candidate data of circRNAs screened by the find_circ software and the candidate data of circRNAs screened by the CIRIexplorer software can greatly reduce the false positives, thereby ensuring the authenticity and accuracy of the screened circRNAs data.

After obtaining the circRNAs data, the present invention extracts reverse splicing reads at a circRNAs looping position in the circRNAs data. In the present invention, the extracting reverse splicing reads at the circRNAs looping position from the obtained circRNAs data is preferably implemented using a samtools view—R instruction in the find_circ software.

After obtaining the reverse splicing reads, the present invention performs single nucleotide variation detection on the reverse splicing reads to obtain SNP loci in the reverse splicing reads. The single nucleotide variation detection is preferably performed using a SNP calling in the GATK software.

After obtaining the SNP loci in the reverse splicing reads, the present invention collects statistics about the number of reads of different genotypes in the reverse splicing reads that are aligned to the SNP loci, to align the proportion of the number of reads of different genotypes to the proportion of the expression quantities of different genotypes.

The method of the present invention can realize high-throughput and high-accuracy analysis of the differential expression of the circRNAs allelic loci through the foregoing steps: provide technical support for the analysis of the allelic expression patterns of subsequent circRNAs, and lay a foundation for comprehensively deciphering the regulation effect of gene plant genome allelic expression and the genetic effect of genomic imprinting. Thus, the present invention has a great application value in the analysis of the genetic effects of plant complex traits and molecular design breeding.

The technical solution provided by the present invention will be described below in detail with reference to examples. However, the examples should not be construed as limiting the protection scope of the present invention.

### EXAMPLE 1

Fresh leaves of P. tomentosa are used. The total RNA is extracted using an RNA extraction kit (MagJ ET Plant RNA Purification Kit, No. K2772). The rRNA is removed using the Ribo-Zero™ rRNA Removal Kits (Plant) (No. MRZPL116) to obtain a Poly (A)-RNA sample. The linear RNA is digested with RNase Rd (the reaction system includes 5 μg of RNA, 5 μL of 10× Reaction Buffer, 20 U of RNase R, and RNase-Free water supplemented to 50 μL) to obtain the Poly (A)-/Ribo-RNA sample. The strand-specific cDNA library is constructed using a SMART kit (SMART cDNA Library Construction Kit, NO. 634901).

Paired-end sequencing is performed using Illumina HiSeq™ 2500 with a sequencing data volume of 12 G. A linker and a redundant sequence are removed; and the transcript is spliced through the default parameters of the Cufflinks software. 20-nt is extracted, with find_circ, at either end of a sequence that is not aligned to a reference sequence (the reference sequence is the populus trichocarpa V3.0 genomic sequence https://phytozome.jgi.doe.gov/pz/portal.html) as a pair of anchor sequences. Each pair of anchor sequences is re-aligned to the reference sequence. If the 5′ terminal of the anchor sequence is aligned to the reference sequence (the starting and terminating loci are denoted as A3 and A4, respectively), and the 3′ terminal of the anchor sequence is aligned to the upstream of a matching locus of the 5′ terminal of the anchor sequence (the starting and ending loci are denoted as A1 and A2, respectively), and a splicing locus (GT-AG) exists between A2 and A3 of the reference sequence, then this read is used as a candidate circRNA. The screening parameters are -q 5, -a 20, -m 2, -d 2, and --noncanonical. The screening criteria are (1) 1-q 5: yjr minimum support number of the anchor sequence alignment is 5; (2) -a 20: the anchor sequence is 20 bp; (3) -m 2: a branch point cannot occur elsewhere in the range of two nucleic acids in the anchor sequence; (4) -d 2: sequence alignment only supports two mismatches; and (5) --noncanonical: GU/AG appears on both sides of a splicing locus and a clear breakpoint can be detected. Moreover, the circRNAs are screened using the default parameters of the CIRIexplorer software. 887 circRNAs are obtained through the find_circ software analysis, and 920 circRNAs are obtained by the CIRIexplorer software. According to the reverse splicing reads of the circRNAs, the intersection of the two prediction results is obtained, and 97 circRNAs are obtained (Table 1).

TABLE 1
Candidate circRNAs of P. tomentosa leaves

| circRNA number | Expression quantity | P value |
|---|---|---|
| pto_circ_0000008 | 699.0198 | 1.52E−143 |
| pto_circ_0000057 | 854.3575 | 3.73E−58 |
| pto_circ_0000187 | 310.6755 | 1.38E−67 |
| pto_circ_0000221 | 1009.695 | 1.29E−102 |
| pto_circ_0000227 | 1087.364 | 6.28E−156 |
| pto_circ_0000284 | 388.3443 | 3.11E−71 |
| pto_circ_0000345 | 466.0132 | 1.62E−72 |
| pto_circ_0000349 | 310.6755 | 4.48E−76 |
| pto_circ_0000521 | 1165.033 | 3.96E−73 |
| pto_circ_0000587 | 310.6755 | 7.09E−60 |
| pto_circ_0000617 | 310.6755 | 7.09E−60 |
| pto_circ_0000674 | 388.3443 | 3.11E−71 |
| pto_circ_0000776 | 621.3509 | 3.01E−77 |
| pto_circ_0000871 | 543.6821 | 2.58E−92 |
| pto_circ_0000892 | 388.3443 | 3.11E−71 |
| pto_circ_0000981 | 310.6755 | 7.09E−60 |
| pto_circ_0001002 | 310.6755 | 7.09E−60 |
| pto_circ_0001090 | 233.0066 | 3.67E−156 |
| pto_circ_0001278 | 310.6755 | 7.09E−60 |
| pto_circ_0001301 | 310.6755 | 7.09E−60 |
| pto_circ_0001411 | 310.6755 | 7.09E−60 |
| pto_circ_0001488 | 310.6755 | 7.09E−60 |
| pto_circ_0001506 | 233.0066 | 8.78E−85 |
| pto_circ_0001508 | 776.6887 | 7.50E−58 |
| pto_circ_0001558 | 699.0198 | 8.32E−112 |
| pto_circ_0001566 | 699.0198 | 2.29E−135 |
| pto_circ_0001622 | 388.3443 | 1.28E−149 |
| pto_circ_0001628 | 2252.397 | 6.33E−149 |
| pto_circ_0001741 | 388.3443 | 3.11E−71 |
| pto_circ_0001816 | 310.6755 | 7.09E−60 |
| pto_circ_0002027 | 466.0132 | 5.21E−82 |
| pto_circ_0002055 | 155.3377 | 7.96E−77 |
| pto_circ_0002058 | 466.0132 | 5.21E−82 |
| pto_circ_0002106 | 621.3509 | 8.89E−168 |
| pto_circ_0002110 | 388.3443 | 3.11E−71 |
| pto_circ_0002122 | 388.3443 | 3.11E−71 |
| pto_circ_0002159 | 1553.377 | 3.33E−203 |
| pto_circ_0002237 | 388.3443 | 1.28E−149 |
| pto_circ_0002256 | 388.3443 | 3.11E−71 |
| pto_circ_0002264 | 1087.364 | 6.28E−156 |
| pto_circ_0002291 | 310.6755 | 7.09E−60 |
| pto_circ_0002292 | 388.3443 | 3.11E−71 |
| pto_circ_0002322 | 310.6755 | 7.09E−60 |
| pto_circ_0002328 | 1242.702 | 3.03E−172 |
| pto_circ_0002377 | 310.6755 | 7.09E−60 |
| pto_circ_0002407 | 2951.417 | 1.60E−191 |
| pto_circ_0002469 | 1631.046 | 2.31E−116 |
| pto_circ_0002472 | 233.0066 | 2.64E−66 |
| pto_circ_0002480 | 621.3509 | 3.15E−102 |
| pto_circ_0002503 | 932.0264 | 8.10E−72 |
| pto_circ_0002610 | 1165.033 | 3.64E−164 |
| pto_circ_0002658 | 233.0066 | 2.64E−66 |
| pto_circ_0002659 | 699.0198 | 8.32E−112 |
| pto_circ_0002730 | 310.6755 | 7.99E−85 |
| pto_circ_0002748 | 155.3377 | 2.47E−130 |
| pto_circ_0002749 | 854.3575 | 3.73E−58 |
| pto_circ_0002750 | 1786.384 | 6.41E−281 |
| pto_circ_0002919 | 233.0066 | 2.09E−75 |
| pto_circ_0002941 | 854.3575 | 3.56E−108 |
| pto_circ_0002982 | 699.0198 | 8.32E−112 |
| pto_circ_0003064 | 155.3377 | 1.27E−97 |
| pto_circ_0003093 | 621.3509 | 3.49E−101 |
| pto_circ_0003157 | 854.3575 | 1.73E−88 |
| pto_circ_0003159 | 310.6755 | 2.20E−59 |
| pto_circ_0003160 | 776.6887 | 4.30E−121 |
| pto_circ_0003167 | 310.6755 | 7.09E−60 |
| pto_circ_0003179 | 621.3509 | 3.15E−102 |
| pto_circ_0003206 | 699.0198 | 8.32E−112 |
| pto_circ_0003256 | 466.0132 | 5.21E−82 |
| pto_circ_0003489 | 155.3377 | 1.27E−97 |
| pto_circ_0003498 | 233.0066 | 2.64E−66 |
| pto_circ_0003579 | 621.3509 | 5.15E−65 |
| pto_circ_0003608 | 1087.364 | 3.49E−101 |
| pto_circ_0003617 | 388.3443 | 3.11E−71 |
| pto_circ_0003620 | 310.6755 | 7.09E−60 |
| pto_circ_0003811 | 543.6821 | 2.58E−92 |
| pto_circ_0003942 | 388.3443 | 3.11E−71 |
| pto_circ_0003970 | 310.6755 | 7.09E−60 |
| pto_circ_0004016 | 1398.04 | 5.61E−188 |
| pto_circ_0004037 | 155.3377 | 3.94E−57 |
| pto_circ_0004047 | 1087.364 | 3.49E−101 |
| pto_circ_0004133 | 2174.728 | 4.16E−247 |
| pto_circ_0004142 | 155.3377 | 8.06E−67 |
| pto_circ_0004203 | 776.6887 | 7.50E−58 |
| pto_circ_0004208 | 543.6821 | 4.88E−83 |
| pto_circ_0004218 | 310.6755 | 7.09E−60 |
| pto_circ_0004238 | 2407.735 | 3.85E−280 |
| pto_circ_0004273 | 543.6821 | 2.58E−92 |
| pto_circ_0004277 | 543.6821 | 2.58E−92 |
| pto_circ_0004278 | 388.3443 | 3.11E−71 |
| pto_circ_0004280 | 2563.073 | 8.51E−198 |
| pto_circ_0004282 | 621.3509 | 3.15E−102 |
| pto_circ_0004284 | 1708.715 | 4.39E−175 |
| pto_circ_0004288 | 543.6821 | 2.64E−82 |
| pto_circ_0004315 | 1242.702 | 3.03E−172 |
| pto_circ_0004318 | 1398.04 | 3.04E−166 |
| pto_circ_0004528 | 699.0198 | 2.19E−74 |

Based on the results of the find_circ analysis, the reverse splicing reads at the circRNAs looping position are extracted using the samtools view—R instruction for subsequent nucleic acid variation analysis.

SNP calling is performed on the extracted reads sequence using the GATK (version: 4.0.1.0) software in the following steps: first, variation detection is performed on two samples by using a HaplotypeCaller tool in the software; --pair-hmm-gap-continuation-penalty parameter is set as 10, and the remaining parameters are set as default values to obtain variation information of each sample; the variation files of each sample are combined by a CombineGVCFs tool; and finally, allelic variation detection is performed on each sample by using a GenotypeGVCFs tool, and a vcf file is generated, where the vcf file contains the mutation loci and genotype information of all samples (Table 2).

The SNPs in the reverse splicing reads are used as markers, and the number of reverse splicing reads on the SNPs is collected and aligned as the expression quantity of the candidate circRNA allelic loci (Table 2).

TABLE 2
Expression patterns of
candidate circRNAs allelic loci of P. tomentosa leaves

| | Reference | Variant | | | |
|---|---|---|---|---|---|
| circRNA number | genotype | genotype | Alt | Ref | Alt/Ref |
| pto_circ_0000057 | G | T | 76 | 124 | 0.612903 |
| pto_circ_0000187 | T | C | 83 | 83 | 1 |
| pto_circ_0000221 | C | T | 705 | 705 | 1 |
| pto_circ_0000227 | A | G | 229 | 47 | 4.87234 |
| pto_circ_0000284 | A | G | 31 | 309 | 0.100324 |
| pto_circ_0000345 | A | G | 14 | 43 | 0.325581 |
| pto_circ_0000349 | C | G | 264 | 264 | 1 |
| pto_circ_0000521 | A | C | 11 | 56 | 0.196429 |
| pto_circ_0000587 | G | A | 82 | 391 | 0.209719 |
| pto_circ_0000617 | G | A | 147 | 229 | 0.641921 |
| pto_circ_0000674 | T | A | 13 | 13 | 1 |
| pto_circ_0000776 | T | C | 49 | 49 | 1 |
| pto_circ_0000871 | T | C | 91 | 415 | 0.219277 |
| pto_circ_0000892 | T | A | 53 | 75 | 0.706667 |
| pto_circ_0000981 | A | G | 91 | 37 | 2.459459 |
| pto_circ_0001002 | T | G | 52 | 857 | 0.060677 |
| pto_circ_0001090 | T | C | 6 | 75 | 0.08 |
| pto_circ_0001278 | G | A | 15 | 86 | 0.174419 |
| pto_circ_0001301 | A | G | 763 | 66 | 11.56061 |
| pto_circ_0001411 | T | A | 66 | 59 | 1.118644 |
| pto_circ_0001488 | G | A | 89 | 949 | 0.093783 |
| pto_circ_0001506 | G | A | 845 | 845 | 1 |
| pto_circ_0001508 | T | A | 95 | 66 | 1.439394 |
| pto_circ_0001558 | G | A | 53 | 53 | 1 |
| pto_circ_0001566 | A | T | 286 | 508 | 0.562992 |
| pto_circ_0001622 | G | A | 3 | 3 | 1 |
| pto_circ_0001628 | G | A | 624 | 624 | 1 |
| pto_circ_0001741 | G | A | 205 | 205 | 1 |
| pto_circ_0001816 | A | G | 984 | 671 | 1.466468 |
| pto_circ_0002027 | T | A | 531 | 65 | 8.169231 |
| pto_circ_0002055 | C | T | 519 | 519 | 1 |
| pto_circ_0002058 | C | T | 92 | 92 | 1 |
| pto_circ_0002106 | T | C | 542 | 228 | 2.377193 |
| pto_circ_0002110 | A | G | 89 | 31 | 2.870968 |
| pto_circ_0002122 | A | G | 45 | 8 | 5.625 |
| pto_circ_0002159 | T | C | 31 | 69 | 0.449275 |
| pto_circ_0002237 | G | T | 31 | 31 | 1 |
| pto_circ_0002256 | G | T | 589 | 589 | 1 |
| pto_circ_0002264 | C | T | 903 | 903 | 1 |
| pto_circ_0002291 | T | A | 438 | 2 | 219 |
| pto_circ_0002292 | T | C | 368 | 71 | 5.183099 |
| pto_circ_0002322 | A | C | 461 | 333 | 1.384384 |
| pto_circ_0002328 | A | T | 182 | 124 | 1.467742 |
| pto_circ_0002377 | T | C | 379 | 69 | 5.492754 |
| pto_circ_0002407 | T | C | 45 | 9 | 5 |
| pto_circ_0002469 | T | C | 69 | 44 | 1.568182 |
| pto_city 0002472 | C | T | 69 | 69 | 1 |
| pto_circ_0002480 | T | C | 9 | 1 | 9 |
| pto_circ_0002503 | C | G | 123 | 123 | 1 |
| pto_circ_0002610 | A | T | 7 | 717 | 0.009763 |
| pto_circ_0002658 | A | G | 42 | 42 | 1 |
| pto_circ_0002659 | T | C | 7 | 8 | 0.875 |
| pto_circ_0002730 | G | A | 8 | 8 | 1 |
| pto_circ_0002748 | T | C | 101 | 427 | 0.236534 |
| pto_circ_0002749 | A | G | 47 | 47 | 1 |
| pto_circ_0002750 | T | A | 47 | 786 | 0.059796 |
| pto_circ_0002919 | C | G | 822 | 822 | 1 |
| pto_circ_0002941 | T | C | 46 | 19 | 2.421053 |
| pto_circ_0002982 | T | A | 85 | 85 | 1 |
| pto_circ_0003064 | C | G | 19 | 19 | 1 |
| pto_circ_0003093 | C | T | 85 | 85 | 1 |
| pto_circ_0003157 | T | A | 123 | 96 | 1.28125 |
| pto_circ_0003159 | A | C | 24 | 24 | 1 |
| pto_circ_0003160 | G | C | 96 | 96 | 1 |
| pto_circ_0003167 | G | T | 879 | 879 | 1 |
| pto_circ_0003179 | T | C | 798 | 798 | 1 |
| pto_circ_0003206 | T | C | 716 | 298 | 2.402685 |
| pto_circ_0003256 | C | G | 612 | 612 | 1 |
| pto_circ_0003489 | T | C | 6 | 24 | 0.25 |
| pto_circ_0003498 | T | C | 298 | 88 | 3.386364 |
| pto_circ_0003579 | G | T | 4 | 4 | 1 |
| pto_circ_0003608 | C | G | 24 | 24 | 1 |
| pto_circ_0003617 | G | A | 88 | 88 | 1 |
| pto_circ_0003620 | A | G | 23 | 23 | 1 |
| pto_circ_0003811 | T | C | 29 | 33 | 0.878788 |
| pto_circ_0003942 | C | G | 29 | 29 | 1 |
| pto_circ_0003970 | G | A | 33 | 33 | 1 |
| pto_circ_0004016 | T | C | 33 | 217 | 0.152074 |
| pto_circ_0004037 | C | T | 356 | 356 | 1 |
| pto_circ_0004047 | A | C | 54 | 54 | 1 |
| pto_circ_0004133 | A | C | 217 | 217 | 1 |
| pto_circ_0004142 | C | T | 135 | 135 | 1 |
| pto_circ_0004203 | G | A | 635 | 635 | 1 |
| pto_circ_0004208 | C | G | 762 | 275 | 2.770909 |
| pto_circ_0004218 | C | T | 542 | 193 | 2.80829 |
| pto_circ_0004238 | C | T | 54 | 437 | 0.12357 |
| pto_circ_0004273 | T | A | 72 | 287 | 0.250871 |
| pto_circ_0004277 | T | C | 27 | 62 | 0.435484 |
| pto_circ_0004278 | C | T | 554 | 74 | 7.486486 |
| pto_circ_0004280 | C | T | 681 | 6 | 113.5 |
| pto_circ_0004282 | G | A | 17 | 17 | 1 |
| pto_circ_0004284 | T | G | 473 | 39 | 12.12821 |
| pto_circ_0004288 | T | C | 972 | 89 | 10.92135 |
| pto_circ_0004315 | A | C | 72 | 72 | 1 |
| pto_circ_0004318 | T | A | 193 | 59 | 3.271186 |
| pto_circ_0004528 | G | A | 275 | 275 | 1 |

The results show that only 44.7% of the circRNA allelic loci in the leaves of P. tomentosa are balanced and the remaining loci are unbalanced.

### EXAMPLE 2

High-temperature treated leaves of P. simonii are used for total RNA extraction using the RNA extraction kit (MagJ ET Plant RNA Purification Kit, No. K2772); and rRNA is removed using the Ribo-Zero™ rRNA Removal Kits (Plant) (No. MRZPL116). Then, the Poly (A) RNAs are combined by using a magnetic bead method, to obtain a Poly (A)-RNA sample; and the linear RNA is digested with RNase Rd (the reaction system includes 5 μg of RNA, 5 μL of 10× Reaction Buffer, 20 U of RNase R, and RNase-Free water supplemented to 50 μL), to obtain a Poly (A)-/Ribo-RNA sample. The strand-specific cDNA library is constructed using a SMART Kit (a SMART cDNA Library Construction Kit, NO. 634901).

Paired-end sequencing is performed using Illumina HiSeq™2500 with a sequencing data volume of 12 G. The linker and the redundant sequence are removed, and the transcript is spliced by the Cufflinks software. 20-nt of anchor sequence is extracted, with find_circ, from either end of the reads that are not aligned to the reference sequence. Each pair of anchor sequences is re-aligned to the reference sequence. If the 5′ terminal of the anchor sequence is aligned to the reference sequence (the starting and terminating locus are denoted as A3 and A4, respectively), and the 3′ terminal of the anchor sequence is aligned to the upstream of this locus (the starting and terminating loci are denoted as A1 and A2, respectively), and a splicing locus (GT-AG) exists between A2 and A3 in the reference sequence, then this read is used as a candidate circRNA. The screening parameters are -h, -v, -s, -G, -n, -p, -q, -a, -m, -d, --noncanonical, --randomize, --allhits, --stranded, --strandpref, and --halfunique. The screening parameters include -q 5, -a 20, -m 2, -d 2, and --noncanonical. The screening criteria of the foregoing parameters are preferably: (1) -q 5: the minimum support number of anchor sequence alignment is 5; (2) -a 20: the anchor sequence is 20 bp; (3) -m 2: a branch point cannot occur elsewhere in the range of two nucleic acids in the anchor sequence; (4) -d 2: sequence alignment only supports two mismatches; and (5) --noncanonical: GU/AG appears on both sides of the splicing locus, and a clear breakpoint can be detected. The circRNAs are screened using the default parameters of the CIRIexplorer software. 804 circRNAs are obtained by the find_circ software analysis, and 670 circRNAs are obtained by the CIRIexplorer software. The intersection of the two prediction results is obtained according to the reverse splicing reads of the circRNAs to obtain 121 circRNAs (Table 3).

TABLE 3
High-temperature response circRNA of P. simonii

| circRNA number | Expression quantity | P value |
|---|---|---|
| psi_circ_0000763 | 3841.211 | 0 |
| psi_circ_0000764 | 4207.041 | 0 |
| psi_circ_0000919 | 4207.041 | 0 |
| psi_circ_0001048 | 0 | 0 |
| psi_circ_0001049 | 0 | 0 |
| psi_circ_0001050 | 0 | 0 |
| psi_circ_0001082 | 3475.381 | 0 |
| psi_circ_0001184 | 0 | 0 |
| psi_circ_0001188 | 182.9148 | 0 |
| psi_circ_0001197 | 731.6593 | 0 |
| psi_circ_0001482 | 2469.35 | 0 |
| psi_circ_0001487 | 5395.987 | 0 |
| psi_circ_0001572 | 3658.296 | 0 |
| psi_circ_0001628 | 3201.009 | 0 |
| psi_circ_0001655 | 3292.467 | 0 |
| psi_circ_0001670 | 7865.337 | 0 |
| psi_circ_0001986 | 4481.413 | 0 |
| psi_circ_0002068 | 7316.593 | 0 |
| psi_circ_0002110 | 4024.126 | 0 |
| psi_circ_0002133 | 1737.691 | 0 |
| psi_circ_0002149 | 25242.24 | 0 |
| psi_circ_0002561 | 11340.72 | 0 |
| psi_circ_0002570 | 3201.009 | 0 |
| psi_circ_0002675 | 731.6593 | 0 |
| psi_circ_0002676 | 640.2018 | 0 |
| psi_circ_0002840 | 3658.296 | 0 |
| psi_circ_0003374 | 7956.794 | 0 |
| psi_circ_0003500 | 5853.274 | 0 |
| psi_circ_0003621 | 4572.87 | 0 |
| psi_circ_0003998 | 3749.754 | 0 |
| psi_circ_0004007 | 3475.381 | 0 |
| psi_circ_0004131 | 4298.498 | 0 |
| psi_circ_0004232 | 2560.807 | 0 |
| psi_circ_0004510 | 7773.88 | 0 |
| psi_circ_0004511 | 17468.36 | 0 |
| psi_circ_0004574 | 1829.148 | 0 |
| psi_circ_0004602 | 2469.35 | 0 |
| psi_circ_0004604 | 0 | 0 |
| psi_circ_0001194 | 914.5741 | 1.7E−302 |
| psi_circ_0001180 | 0 | 1.9E−297 |
| psi_circ_0004576 | 2377.893 | 6.9E−297 |
| psi_circ_0001146 | 2377.893 | 4.1E−289 |
| psi_circ_0000292 | 2286.435 | 1.4E−288 |
| psi_circ_0001987 | 2286.435 | 1.4E−288 |
| psi_circ_0001765 | 1829.148 | 1.1E−275 |
| psi_circ_0000460 | 1554.776 | 1.3E−275 |
| psi_circ_0001416 | 2103.52 | 1.1E−271 |
| psi_circ_0000295 | 2012.063 | 4.4E−263 |
| psi_circ_0001775 | 2012.063 | 4.4E−263 |
| psi_circ_0001977 | 1920.606 | 2.4E−254 |
| psi_circ_0004235 | 1737.691 | 7.3E−252 |
| psi_circ_0000760 | 1737.691 | 1.8E−236 |
| psi_circ_0004005 | 1737.691 | 1.8E−236 |
| psi_circ_0004036 | 1737.691 | 1.8E−236 |
| psi_circ_0000180 | 0 | 5.7E−230 |
| psi_circ_0002557 | 1646.233 | 1.9E−228 |
| psi_circ_0001020 | 1646.233 | 2.5E−227 |
| psi_circ_0003449 | 1646.233 | 2.5E−227 |
| psi_circ_0004601 | 1646.233 | 2.5E−227 |
| psi_circ_0000308 | 1554.776 | 5E−218 |
| psi_circ_0001470 | 1554.776 | 5E−218 |
| psi_circ_0001940 | 1554.776 | 5E−218 |
| psi_circ_0002839 | 1554.776 | 5E−218 |
| psi_circ_0004453 | 2286.435 | 3.4E−217 |
| psi_circ_0004299 | 0 | 1.5E−215 |
| psi_circ_0001346 | 1463.319 | 1.5E−208 |
| psi_circ_0004247 | 1463.319 | 1.5E−208 |
| psi_circ_0004367 | 1463.319 | 1.5E−208 |
| psi_circ_0001228 | 1554.776 | 1.7E−205 |
| psi_circ_0002735 | 0 | 9.6E−201 |
| psi_circ_0000311 | 1371.861 | 6.5E−199 |
| psi_circ_0000476 | 1371.861 | 6.5E−199 |
| psi_circ_0004006 | 1371.861 | 6.5E−199 |
| psi_circ_0000787 | 1280.404 | 4.5E−189 |
| psi_circ_0003832 | 1280.404 | 4.5E−189 |
| psi_circ_0003942 | 1280.404 | 4.5E−189 |
| psi_circ_0004454 | 1280.404 | 4.5E−189 |
| psi_circ_0004456 | 1280.404 | 4.5E−189 |
| psi_circ_0001134 | 1188.946 | 5E−179 |
| psi_circ_0003450 | 1188.946 | 5E−179 |
| psi_circ_0004035 | 1188.946 | 5E−179 |
| psi_circ_0004575 | 1188.946 | 5E−179 |
| psi_circ_0001566 | 1188.946 | 1.8E−176 |
| psi_circ_0001182 | 0 | 9.1E−170 |
| psi_circ_0002674 | 0 | 9.1E−170 |
| psi_circ_0003631 | 0 | 9.1E−170 |
| psi_circ_0000368 | 1097.489 | 9.6E−169 |
| psi_circ_0001874 | 1097.489 | 9.6E−169 |
| psi_circ_0003244 | 1097.489 | 9.6E−169 |
| psi_circ_0003656 | 1097.489 | 9.6E−169 |
| psi_circ_0003929 | 1097.489 | 9.6E−169 |
| psi_circ_0002987 | 182.9148 | 3.8E−161 |
| psi_circ_0001046 | 1006.031 | 3.3E−158 |
| psi_circ_0001129 | 1006.031 | 3.3E−158 |
| psi_circ_0001133 | 1006.031 | 3.3E−158 |
| psi_circ_0001139 | 1006.031 | 3.3E−158 |
| psi_circ_0002968 | 1006.031 | 3.3E−158 |
| psi_circ_0003452 | 1006.031 | 3.3E−158 |
| psi_circ_0004248 | 1006.031 | 3.3E−158 |
| psi_circ_0003699 | 0 | 1.8E−153 |
| psi_circ_0001217 | 1097.489 | 6.1E−153 |
| psi_circ_0001568 | 1097.489 | 6.1E−153 |
| psi_circ_0003419 | 1554.776 | 5.4E−152 |
| psi_circ_0000367 | 914.5741 | 2.2E−147 |
| psi_circ_0000789 | 914.5741 | 2.2E−147 |
| psi_circ_0000790 | 914.5741 | 2.2E−147 |
| psi_circ_0001130 | 914.5741 | 2.2E−147 |
| psi_circ_0001211 | 914.5741 | 2.2E−147 |
| psi_circ_0001961 | 914.5741 | 2.2E−147 |
| psi_circ_0002736 | 914.5741 | 2.2E−147 |
| psi_circ_0003385 | 914.5741 | 2.2E−147 |
| psi_circ_0004034 | 914.5741 | 2.2E−147 |
| psi_circ_0000989 | 274.3722 | 2.2E−144 |
| psi_circ_0002909 | 1280.404 | 6.2E−140 |
| psi_circ_0003015 | 1280.404 | 6.2E−140 |
| psi_circ_0000010 | 0 | 1.7E−136 |
| psi_circ_0003066 | 0 | 1.7E−136 |
| psi_circ_0000004 | 823.1167 | 3E−136 |
| psi_circ_0000202 | 823.1167 | 3E−136 |
| psi_circ_0000575 | 823.1167 | 3E−136 |
| psi_circ_0001159 | 823.1167 | 3E−136 |

The reverse splicing reads data tag files at the circRNAs looping position are sorted according to the result of the find_circ analysis, and the reverse splicing reads at the circRNAs looping position are extracted using the samtools view—R instruction for subsequent nucleic acid variation analysis.

SNP calling is performed on the extracted reads sequence using the GATK (version: 4.0.1.0) software in the following steps: first, variation detection is performed on two samples by using a HaplotypeCaller tool, --pair-hmm-gap-continuation-penalty parameter is set as 10, and the remaining parameters are default values to obtain variation information of each sample; the variation files of each sample are combined by a CombineGVCFs tool; and finally, allelic variation detection is performed on each sample by using a GenotypeGVCFs tool, and a vcf file is generated, where the vcf file contains the mutation loci and genotype information of all samples (Table 4).

The SNPs in the reverse splicing reads are used as markers, the number of reverse splicing reads on the SNPs is collected and aligned as the expression quantity of the candidate circRNA allelic loci (Table 4).

TABLE 4
Expression pattern of high-temperature response
circRNA allelic loci of P. simonii

| | Reference | Variant | | | |
|---|---|---|---|---|---|
| circRNA number | genotype | genotype | Alt | Ref | Alt/Ref |
| psi_circ_0000004 | A | C | 24 | 786 | 0.030534 |
| psi_circ_0000010 | C | T | 85 | 857 | 0.099183 |
| psi_circ_0000180 | A | G | 47 | 75 | 0.626667 |
| psi_circ_0000202 | G | C | 96 | 949 | 0.101159 |
| psi_circ_0000292 | A | T | 182 | 124 | 1.467742 |
| psi_circ_0000295 | T | C | 9 | 1 | 9 |
| psi_circ_0000308 | C | G | 19 | 309 | 0.061489 |
| psi_circ_0000311 | G | T | 4 | 437 | 0.009153 |
| psi_circ_0000367 | C | G | 123 | 217 | 0.56682 |
| psi_circ_0000368 | T | A | 72 | 287 | 0.250871 |
| psi_circ_0000460 | T | C | 69 | 44 | 1.568182 |
| psi_circ_0000476 | C | G | 24 | 86 | 0.27907 |
| psi_circ_0000575 | G | T | 879 | 56 | 15.69643 |
| psi_circ_0000760 | T | C | 7 | 8 | 0.875 |
| psi_circ_0000763 | G | T | 76 | 124 | 0.612903 |
| psi_circ_0000764 | T | C | 83 | 391 | 0.212276 |
| psi_circ_0000787 | A | G | 23 | 415 | 0.055422 |
| psi_circ_0000789 | A | T | 7 | 717 | 0.009763 |
| psi_circ_0000790 | A | G | 42 | 427 | 0.098361 |
| psi_circ_0000919 | C | T | 705 | 427 | 1.651054 |
| psi_circ_0000989 | T | C | 46 | 19 | 2.421053 |
| psi_circ_0001020 | C | G | 822 | 24 | 34.25 |
| psi_circ_0001046 | T | C | 972 | 89 | 10.92135 |
| psi_circ_0001048 | A | G | 229 | 47 | 4.87234 |
| psi_circ_0001049 | A | G | 31 | 309 | 0.100324 |
| psi_circ_0001050 | A | G | 14 | 43 | 0.325581 |
| psi_circ_0001082 | C | G | 264 | 287 | 0.919861 |
| psi_circ_0001129 | A | C | 72 | 43 | 1.674419 |
| psi_circ_0001130 | T | C | 7 | 8 | 0.875 |
| psi_circ_0001133 | T | A | 193 | 59 | 3.271186 |
| psi_circ_0001134 | C | T | 356 | 62 | 5.741935 |
| psi_circ_0001139 | G | A | 275 | 69 | 3.985507 |
| psi_circ_0001146 | A | C | 461 | 333 | 1.384384 |
| psi_circ_0001159 | T | C | 798 | 508 | 1.570866 |
| psi_circ_0001180 | T | A | 438 | 2 | 219 |
| psi_circ_0001182 | C | G | 762 | 275 | 2.770909 |
| psi_circ_0001184 | A | C | 11 | 56 | 0.196429 |
| psi_circ_0001188 | G | A | 82 | 391 | 0.209719 |
| psi_circ_0001194 | C | T | 903 | 124 | 7.282258 |
| psi_circ_0001197 | G | A | 147 | 229 | 0.641921 |
| psi_circ_0001211 | G | A | 8 | 229 | 0.034934 |
| psi_circ_0001217 | T | C | 69 | 44 | 1.568182 |
| psi_circ_0001228 | T | C | 6 | 24 | 0.25 |
| psi_circ_0001346 | T | C | 798 | 75 | 10.64 |
| psi_circ_0001416 | C | T | 69 | 8 | 8.625 |
| psi_circ_0001470 | C | T | 85 | 8 | 10.625 |
| psi_circ_0001482 | T | A | 13 | 33 | 0.393939 |
| psi_circ_0001487 | T | C | 49 | 49 | 1 |
| psi_circ_0001566 | G | A | 635 | 635 | 1 |
| psi_circ_0001568 | C | T | 69 | 69 | 1 |
| psi_circ_0001572 | T | C | 91 | 415 | 0.219277 |
| psi_circ_0001628 | T | A | 53 | 75 | 0.706667 |
| psi_circ_0001655 | A | G | 91 | 37 | 2.459459 |
| psi_circ_0001670 | T | G | 52 | 857 | 0.060677 |
| psi_circ_0001765 | T | C | 45 | 9 | 5 |
| psi_circ_0001775 | C | G | 123 | 123 | 1 |
| psi_circ_0001874 | T | C | 27 | 62 | 0.435484 |
| psi_circ_0001940 | T | A | 123 | 96 | 1.28125 |
| psi_circ_0001961 | T | C | 101 | 427 | 0.236534 |
| psi_circ_0001977 | A | T | 7 | 717 | 0.009763 |
| psi_circ_0001986 | T | C | 6 | 75 | 0.08 |
| psi_circ_0001987 | T | C | 379 | 69 | 5.492754 |
| psi_circ_0002068 | G | A | 15 | 86 | 0.174419 |
| psi_circ_0002110 | A | G | 763 | 66 | 11.56061 |
| psi_circ_0002133 | T | A | 66 | 59 | 1.118644 |
| psi_circ_0002149 | G | A | 89 | 949 | 0.093783 |
| psi_circ_0002557 | T | A | 47 | 786 | 0.059796 |
| psi_circ_0002561 | G | A | 845 | 845 | 1 |
| psi_circ_0002570 | T | A | 95 | 66 | 1.439394 |
| psi_circ_0002674 | C | T | 542 | 193 | 2.80829 |
| psi_circ_0002675 | G | A | 53 | 53 | 1 |
| psi_circ_0002676 | A | T | 286 | 508 | 0.562992 |
| psi_circ_0002735 | T | C | 298 | 88 | 3.386364 |
| psi_circ_0002736 | A | G | 47 | 47 | 1 |
| psi_circ_0002839 | A | C | 24 | 24 | 1 |
| psi_circ_0002840 | G | A | 3 | 3 | 1 |
| psi_circ_0002909 | T | A | 85 | 85 | 1 |
| psi_circ_0002968 | A | C | 461 | 333 | 1.384384 |
| psi_circ_0002987 | T | G | 473 | 39 | 12.12821 |
| psi_circ_0003015 | C | G | 19 | 19 | 1 |
| psi_circ_0003066 | T | A | 123 | 96 | 1.28125 |
| psi_circ_0003244 | C | T | 554 | 74 | 7.486486 |
| psi_circ_0003374 | G | A | 624 | 624 | 1 |
| psi_circ_0003385 | T | A | 47 | 786 | 0.059796 |
| psi_circ_0003419 | T | C | 9 | 1 | 9 |
| psi_circ_0003449 | T | C | 46 | 19 | 2.421053 |
| psi_circ_0003450 | A | C | 54 | 54 | 1 |
| psi_circ_0003452 | A | T | 182 | 124 | 1.467742 |
| psi_circ_0003500 | G | A | 205 | 205 | 1 |
| psi_circ_0003621 | A | G | 984 | 671 | 1.466468 |
| psi_circ_0003631 | C | T | 54 | 437 | 0.12357 |
| psi_circ_0003656 | C | T | 681 | 6 | 113.5 |
| psi_circ_0003699 | T | C | 45 | 9 | 5 |
| psi_circ_0003832 | T | C | 29 | 33 | 0.878788 |
| psi_circ_0003929 | G | A | 17 | 17 | 1 |
| psi_circ_0003942 | C | G | 29 | 29 | 1 |
| psi_circ_0003998 | T | A | 531 | 65 | 8.169231 |
| psi_circ_0004005 | G | A | 8 | 8 | 1 |
| psi_circ_0004006 | G | A | 88 | 88 | 1 |
| psi_circ_0004007 | C | T | 519 | 519 | 1 |
| psi_circ_0004034 | C | G | 822 | 822 | 1 |
| psi_circ_0004035 | A | C | 217 | 217 | 1 |
| psi_circ_0004036 | T | C | 101 | 427 | 0.236534 |
| psi_circ_0004131 | C | T | 92 | 92 | 1 |
| psi_circ_0004232 | T | C | 542 | 228 | 2.377193 |
| psi_circ_0004235 | A | G | 42 | 42 | 1 |
| psi_circ_0004247 | T | C | 716 | 298 | 2.402685 |
| psi_circ_0004248 | T | C | 379 | 69 | 5.492754 |
| psi_circ_0004299 | G | T | 879 | 879 | 1 |
| psi_circ_0004367 | C | G | 612 | 612 | 1 |
| psi_circ_0004453 | G | C | 96 | 96 | 1 |
| psi_circ_0004454 | G | A | 33 | 33 | 1 |
| psi_circ_0004456 | T | C | 33 | 217 | 0.152074 |
| psi_circ_0004510 | A | G | 89 | 31 | 2.870968 |
| psi_circ_0004511 | A | G | 45 | 8 | 5.625 |
| psi_circ_0004574 | T | C | 31 | 69 | 0.449275 |
| psi_circ_0004575 | C | T | 135 | 135 | 1 |
| psi_circ_0004576 | T | C | 368 | 71 | 5.183099 |
| psi_circ_0004601 | T | A | 85 | 85 | 1 |
| psi_circ_0004602 | G | T | 31 | 31 | 1 |
| psi_circ_0004604 | G | T | 589 | 589 | 1 |

The results show that only 25.8% of the circRNA allelic loci in the leaf tissues of P. simonii treated by high temperature stress are of balance expression, and the remaining loci are of imbalance expression.

It can be seen from the above examples that the method provided by the present invention uses strand-specific library RNA sequencing, combines with circRNA analysis software and nucleic acid variation analysis software, to accurately analyze the expression pattern of plant circRNA allelic loci in a high-throughput manner. It can be seen from the results in the examples that the method can achieve high-throughput analysis of allelic expression of plant circRNAs, and provides a new research approach for systematically analyzing the allelic expression pattern of the circRNAs by fully utilizing the results of transcriptome sequencing.

The foregoing descriptions are only preferred implementation manners of the present invention. It should be noted that for a person of ordinary skill in the art, several improvements and modifications may further be made without departing from the principle of the present invention. These improvements and modifications should also be deemed as falling within the protection scope of the present invention.

What is claimed is:

1. A method for high-throughput detection of differential expression of plant circRNA allelic loci, comprising the following steps:
1) extracting total RNA of a plant sample, and constructing a strand-specific library with the total RNA;
2) performing paired-end sequencing on the strand-specific library in step 1) with Illumina HiSeq to obtain raw sequencing data;
3) screening circRNAs data from the raw sequencing data obtained in step 2);
4) extracting reverse splicing reads at a circRNAs looping position from the circRNAs data obtained in step 3);
5) performing single nucleotide variation detection on the reverse splicing reads to obtain SNP loci in the reverse splicing reads; and
6) collecting statistics about the number of reads of different genotypes, which are aligned to the SNP loci in step 5, in the reverse splicing reads in step 4, to align the proportion of the number of reads of different genotypes to the proportion of expression quantities of different genotypes.

2. The method according to claim 1, wherein the screening circRNAs data in step 3) comprises the following steps:
3.1) performing transcript splicing on the raw sequencing data according to a reference genome;
3.2) extracting 18-22 nt from both ends of each read in the raw sequencing data that is not aligned to the reads on the reference genome, and forming a pair of anchor sequences, the anchor sequence comprising a 5′-terminal sequence and a 3′-terminal sequence; and
3.3) re-aligning the anchor sequence to a reference genome, wherein the 5′-terminal sequence of the anchor sequence is aligned to the 3′-terminal of the reference sequence, and the 3′-terminal sequence of the anchor sequence is aligned to the upstream of a matching locus of the 5′-terminal sequence of the anchor sequence in the reference sequence, and there is a splicing locus GT-AG exists between the matching locus of the 5′-terminal sequence of the anchor sequence and a matching locus of the 3′-terminal sequence of the anchor sequence in the reference sequence, and this read is used as circRNA data.

3. The method according to claim 1, wherein the screening circRNAs data is implemented by means of find_circ software and CIRIexplorer software.

4. The method according to claim 2, wherein the screening circRNAs data is implemented by means of find_circ software and CIRIexplorer software.

5. The method according to claim 3, wherein the circRNAs are screened by using the find_circ software and the CIRIexplorer software, respectively, to obtain candidate data of circRNAs screened by the find_circ software and candidate data of circRNAs screened by the CIRIexplorer software, and an intersection of the candidate data of circRNAs screened by the find_circ software and the candidate data of circRNAs screened by the CIRIexplorer software is used as circRNAs data.

6. The method according to claim 4, wherein the circRNAs are screened by using the find_circ software and the CIRIexplorer software, respectively, to obtain candidate data of circRNAs screened by the find_circ software and candidate data of circRNAs screened by the CIRIexplorer software, and an intersection of the candidate data of circRNAs screened by the find_circ software and the candidate data of circRNAs screened by the CIRIexplorer software is used as circRNAs data.

7. The method according to claim 1, wherein the extracting reverse splicing reads at a circRNAs looping position in the circRNAs obtained in step 4) is implemented using a samtools view—R instruction in the find_circ software.

8. The method according to claim 1, wherein the single nucleotide variation detection in step 5) is performed using a SNP calling in GATK software.

9. The method according to claim 1, wherein after extracting total RNA of a plant sample and before constructing a strand-specific library in step 1), the method further comprises a step of removing rRNA and a step of linear RNA digestion that are performed sequentially.

10. The method according to claim 9, wherein a reaction system of the linear RNA digestion is 50 μL, comprising the following components: 5 μg of RNA, 5 μL of 10× Reaction Buffer, 20 U of RNase R, and the balance of RNase-Free water.

11. The method according to claim 9, wherein the temperature for the linear RNA digestion is 36-38° C., and the time for the linear RNA digestion is 1-2 h.

12. The method according to claim 10, wherein the temperature for the linear RNA digestion is 36-38° C., and the time for the linear RNA digestion is 1-2 h.

13. The method according to claim 1, wherein the plant is a forest tree.
